(19) United States
(12) Patent Application Publication
(10) Pub. No.: US 2020/0397981 A1
(43) Pub. Date: Dec. 24, 2020

(54) Compression-Activated Refillable Pump for Controlled Drug Delivery

(71) Applicant: Cylerus, Inc., Melbourne, FL (US)

(72) Inventor: Stephen R. HANSON, Edmonds, WA (US)

(21) Appl. No.: 16/905,176

(22) Filed: Jun. 18, 2020

Related U.S. Application Data
(60) Provisional application No. 62/863,553, filed on Jun. 19, 2019.

(51) Int. Cl. A61M 5/145; A61M 5/142; A61M 5/168; A61M 5/148

(52) U.S. Cl. CPC A61M 5/1454; A61M 5/14276; A61M 5/16881; A61M 5/14224; A61K 31/436; A61M 5/14248; A61M 5/148; A61M 2005/14296; A61M 2202/0007; A61M 5/16827

(57) ABSTRACT

Refillable compression-activated pump devices having a first reservoir, a second reservoir connected by a one-way valve and methods of using the pump devices are provided. The compression-activated pumps can be filled with fluid containing a drug, pressurized by compressing the first or second reservoir to deliver the drug to another device, and refilled when drug is depleted.

### CROSS-REFERENCE TO RELATED APPLICATIONS

This application claims the benefit of U.S. Provisional Patent Application No. 62/863,553; filed Jun. 19, 2019, which is hereby incorporated by reference in its entirety.

All references cited herein, including but not limited to patents and patent applications, are incorporated by reference in their entirety.

### BACKGROUND

Local drug delivery in the body remains a challenging problem. While efficacious drugs have been identified and characterized, controlled delivery of such drugs at a sufficient concentration for a sufficient amount of time while avoiding detrimental systemic side effects remains elusive. Pumps are routinely used for local and short-term drug delivery in the body. Small, powered pumps (e.g., for infusing insulin, chemotherapy, analgesics) can be implanted near the site of treatment and deliver a drug at a desired rate and in a desired amount. However, such pumps are complex, require replacement of a power source (e.g., battery) or replacement entirely, and are prone to malfunction.

Blood pumps can also be used to preserve the functions of blood conduits. Blood conduits may be constructed of either native arteries or veins, or synthetic materials such as expanded polytetrafluoroethylene (ePTFE) graft material, all of which are frequently used in vascular surgery. Vascular grafts are commonly employed for the creation of arteriovenous (A-V) access used as needle insertion sites to enable blood removal and return for hemodialysis that is performed 2-3 times per week in patients with end stage renal disease (ESRD). More than 75,000 new hemodialysis grafts are placed in the U.S. each year and costs for creating and maintaining these grafts exceed $1 billion annually.

Vascular grafts are also indicated in the treatment of peripheral vascular disease (PVD) that is the result of atherosclerosis causing arterial obstruction with pain and cramping in the legs, especially below the knee where vessels are smaller. A blood conduit, such as ePTFE or native vein, is often used to bypass the obstructed artery. The durability and long-term patency of blood conduits used to replace diseased arteries in PVD are substantially better than results with ePTFE grafts used to provide chronic blood access for hemodialysis.

Over 80% of arteriovenous access grafts and 20% of peripheral arterial bypass grafts will fail or become dysfunctional within one year after implantation resulting in considerable patient morbidity and substantial costs to the healthcare system. Graft failure is often due to neointimal hyperplasia (i.e., obstructive tissue ingrowth) at the venous outflow tract that is caused by mechanical injury to blood vessels. While drugs that inhibit vascular neointimal hyperplasia in these settings are available, delivery of these drugs to the site of injury, at a safe yet effective dose, for a sufficient period of time, has been challenging.

U.S. Pat. No. 5,399,352 ('352 patent) is directed to placing drug(s) in an external cuff-reservoir for delivery of drug(s) across the wall of the vascular graft to the graft luminal surface. Tubing may be attached to permit refilling or changing of the drug. The '352 patent also refers to use of a device such as a pump to create positive pressure and provide constant controlled drug delivery. However, if the size of the pores in the graft material vary, drug delivery will be non-uniform, and neointimal hyperplasia will not be adequately inhibited.

U.S. Pat. No. 8,721,711 ('711 patent) refers to the use of a microporous membrane within the graft cuff-reservoir where the pore size can be selected to provide more uniform drug delivery. U.S. Pat. No. 8,808,255 ('255 patent) provides for a drug delivery cuff-reservoir that can be removed or repositioned.

However, the drug delivery devices of the '352, '711, and '255 patents are limited because they each require use of an optionally refillable powered pump to achieve prolonged and steady rates of drug delivery. Such pumps are expensive, require expensive approval processes, and are typically bulky and therefore uncomfortable and inconvenient for patient use. Without a pump to control delivery of the drug to a cuff-reservoir, delivery of solution-phase drug from a graft cuff-reservoir would typically decrease exponentially over a relatively short period of time.

Therefore, suitable refillable pumps are needed that deliver drug reliably without the need for battery power and are optionally inexpensive, clinically approved, and patient appropriate.

### SUMMARY

Aspects described herein provide refillable, controlled delivery devices for vascular delivery of drugs to blood vessels or to the luminal surface of a vascular graft. In one aspect, a refillable, pressure activated or compression pump for controlled delivery of a fluid and methods of using the same is provided. In these aspects, the pump can comprise a first reservoir, a second reservoir, a one-way valve, an inlet port, and an outlet port. The first reservoir and the second reservoir can be fluidly connected via the one-way valve, the first reservoir can include an inlet port and a first pressure receiver, and the second reservoir can include a second pressure receiver and is fluidly connected to the outlet port.

Yet further aspects provide methods for controlled delivery of a fluid by providing a pump having a first reservoir, a second reservoir, a one-way valve, an inlet port, and an outlet port. In this aspect, the first reservoir and the second reservoir are fluidly connected via the one-way valve, the first reservoir comprises an inlet port and a first pressure receiver, and the second reservoir comprises a second pressure receiver fluidly connected to the outlet port. In this aspect, the first reservoir is filled with a fluid, the fluid flows into the second reservoir from the first reservoir via the one-way valve, and is delivered through the outlet port.

### BRIEF DESCRIPTION OF THE DRAWINGS

FIG. 1A illustrates an exemplary elastic refillable, pressure-activated pump for controlled delivery of a fluid prior to filling with a drug;

FIG. 1B illustrates the elastic pressure-activated pump of FIG. 1A while drug is delivered to a patient;

FIG. 1C illustrates the elastic pressure-activated pump of FIG. 1B when pressure force (F) is applied to the pump to restore pressure in the pump;

FIG. 2A illustrates an exemplary non-elastic, collapsible, refillable, pressure-activated pump for controlled delivery of a fluid prior to filling with a drug;

FIG. 2B illustrates the non-elastic, collapsible, pressure-activated pump of FIG. 2A while drug is delivered to a patient;

FIG. 2C illustrates the non-elastic, collapsible, pressure-activated pump of FIG. 2B when pressure force (F) is applied to the pump to restore pressure in the pump;

FIG. 3A illustrates the use of a non-elastic collapsible, refillable reservoir combined with an elastic reservoir prior to delivery of drug to a patient;

FIG. 3B illustrates the use of the non-elastic collapsible, refillable reservoir in combination with an elastic reservoir of FIG. 3A after drug is pushed from the first reservoir to the second reservoir;

FIG. 3C illustrates an exemplary fill stop and a second reservoir contained within an optional elastic membrane or collapsible membrane element;

FIG. 3D illustrates alternative aspects of the optional elastic membrane or expandable-collapsible element of FIG. 3C;

FIG. 4 illustrates an exemplary aspect of the compression-activated refillable pump;

FIG. 5A illustrates an exemplary subcutaneous drug pump in flat orientation with a substantially circular shape;

FIG. 5B illustrates an exemplary subcutaneous drug pump with force applied axially (top panel) and longitudinally (bottom panel);

FIG. 6 illustrates an exemplary drug pump with force applied axially to pressurize and expand the second reservoir, and an exemplary spring for compressing the second reservoir to deliver the drug radially;

FIG. 7A shows a top view of an exemplary subcutaneous drug pump with force applied longitudinally; and

FIG. 7B shows a side view of the exemplary drug pump of FIG. 7A before during and after applying a longitudinal force to the drug pump.

### DETAILED DESCRIPTION

Before describing exemplary aspects described herein, it is to be understood that the invention is not limited to the details of construction or process steps set forth in the following description. The aspects described herein are capable of being practiced or being carried out in various ways.

Aspects described herein provide a refillable, compression-activated pump for delivery of a fluid comprising a first reservoir, a second reservoir, a one-way valve, an inlet port, and an outlet port. In this aspect, the first reservoir and the second reservoir are fluidly connected via the one-way valve, the first reservoir comprising the inlet port and a first pressure receiver, and the second reservoir comprising a second pressure receiver and the outlet port. In this aspect, the first reservoir and the second reservoir are fluidly connected via the one-way valve, the first reservoir comprises an inlet port and a first pressure receiver, and the second reservoir comprises a second pressure receiver and is fluidly connected to the outlet port.

It is to be understood that the refillable, compression-activated pump can be used to deliver any fluid, including a drug, to any portion of the body or prosthetic device in need of treatment (e.g., vascular graft, skin, blood, muscle tissue, organ, etc.). In another aspect, the compression-activated pump can be used to deliver drugs that are delivered over a long period of time (e.g., pain medication, insulin, anti-inflammatories, etc.). In a further aspect, the compression-activated pump can optionally include one or more sensors to monitor rate and volume of drug delivery and send data to a wireless device via a wireless protocol (e.g., bluetooth).

The term “fluidly connected” as used herein means that fluid can move from one part to another part without substantially leaking and, if desired, without being exposed to non-sterile or ambient conditions. The first reservoir and the second reservoir can each comprise an elastic material (e.g., silicone rubber) or a non-elastic material (e.g., polytetrafluoroethylene, polyethylene). The non-elastic material can be a non-elastic collapsible material.

It is understood that the first and second reservoirs can both be elastic, both be non-elastic, or one can be elastic while the other in non-elastic. The term “elastic” refers to a material that is able to resume its normal shape spontaneously after stretching, contraction, dilatation, or distortion.

The term “pressure receiver” refers to an element or part that can receive and transfer compression or applied pressure (force per unit area) from an external source (e.g., finger, spring, magnet etc.) into pressure energy (i.e., energy within the fluid volume) thereby increasing, for example, pressure in a sealed unit. A pressure receiver can be pressed or compressed one or more times to generate pressure energy.

The term “reservoir” refers to an article or device capable of holding or retaining fluid. Examples of pump reservoirs include, but are not limited to, a bladder, chamber, etc.

The term “valve” refers to a device which controls or limits the passage of fluid. The term “one-way valve” refers to a valve which permits fluid to flow in one direction. For example, fluid is permitted to flow from the first reservoir to the second reservoir but not from the second reservoir to the first reservoir. Examples of one-way valves include, but are not limited to, check valve, clack valve, non-return valve, reflux valve, retention valve.

The term “port” refers to a diaphragm, conduit or connector from outside of the pump into the pump, from inside the exemplary pump to outside of the pump, or between portions or parts of the pump. For example, the “inlet port” is a conduit to introduce fluid into the first reservoir.

In another aspect, the first reservoir can be filled with a fluid through the inlet port, causing the fluid to move through the one-way valve into the second reservoir. In yet another aspect, the fluid from the second reservoir can be delivered through the outlet port into, for example, a patient. The outlet port can be configured to connect to a catheter, port or another device to deliver fluid to a patient.

In another aspect, the fluid volume in the first reservoir can be increased by supplying fluid through the inlet port. In a further aspect, increasing the pressure in the first reservoir by applying external pressure to the first pressure receiver causes fluid to move through the one-way valve into the second reservoir.

In a further aspect, increasing pressure in the first reservoir applies pressure to the second pressure receiver, increasing pressure in the second reservoir. In yet another aspect, the pressure in the first reservoir can be increased when the pressure in the second reservoir is below a threshold sufficient to deliver fluid from the second reservoir at a desired rate.

In one aspect, the first pressure receiver is a first flexible membrane and the second pressure receiver is a second flexible membrane. The first flexible membrane can comprise a wall of the first reservoir, and the second flexible membrane can comprise a wall of the second reservoir.

In a further aspect, manual compression force can optionally be applied to the first flexible elastic or inelastic collapsible pressure receiver, and compression force can be applied to the first or the second flexible pressure receiver by one or more forces derived from, for example, elastic membrane contraction, spring expansion, magnetic repulsion, compressed gas expansion, spring contraction, and magnetic attraction or other forces applied to a flexible membrane.

In yet another aspect, the pump can further comprise a spring engaged with the second reservoir. The term “spring” as used herein refers to any elastic member capable of deforming under the action of a load or force and recovering to its original shape when the load is removed (e.g., a helical spring, a torsional spring, and a lever spring). In this aspect, a spring can be made of any suitable material (e.g., metal or plastic). A spring can store potential energy and, when the load is removed, release kinetic energy.

In one aspect, increasing a pressure in the first reservoir by applying external pressure to the first pressure receiver lengthens the spring, and increases the volume of the second reservoir, wherein the fluid moves through the one-way valve into the second reservoir, and wherein when force is applied to the second pressure receiver by a retraction of the spring, the fluid moves from the second reservoir into the outlet port.

The initial length of the spring can be about equal to the diameter of the pump. In a further aspect, the volume of the second reservoir can be increased by about 10 to about 20% after applying external pressure to the first pressure receiver.

In yet another aspect, the pump further comprises a compression band disposed around the second reservoir. In this aspect, the term “compression band” refers to a substantially circular member capable of deforming under the action of a load or force and recovering to its original shape when the load is removed (e.g., circular spiral spring, a compression spring, and a constant force spring). A compression band can also be form of a spring.

In this aspect, increasing a pressure in the first reservoir by applying external pressure to the first pressure receiver increases a circumference of the compression band and increases the volume of the second reservoir, wherein the fluid moves through the one-way valve into the second reservoir, and wherein force is applied to the second pressure receiver by a retraction of the compression band, causing the fluid to be delivered through the outlet port from the second reservoir.

In another aspect, the initial circumference of the compression band can be about equal to the diameter of the pump. In another aspect, the volume of the second reservoir is increased by about 10 to about 20% after applying external pressure to the first pressure receiver.

In one aspect, the pump further comprises a refill port associated with the first reservoir for refilling the pump with the fluid. The fluid can comprise a drug (e.g., an olimus drug comprising at least one of sirolimus, everolimus, zotarolimus, tacrolimus, pimecrolimus, temsirolimus, ridaforolimus or biolimus).

Further aspects provide a kit comprising the pump aspects described herein, and a container (e.g., vial, an ampule, a capsule, and a syringe) comprising a drug (e.g., an olimus drug comprising at least one of sirolimus, everolimus, zotarolimus, tacrolimus, pimecrolimus, temsirolimus, ridaforolimus or biolimus).

Methods of delivering a fluid are provided by (1) providing a pump having a first reservoir, a second reservoir, a one-way valve, an inlet port, and an outlet port, wherein the first reservoir and the second reservoir are fluidly connected via the one-way valve, the first reservoir comprises the inlet port and a first pressure receiver, and the second reservoir comprises a second pressure receiver fluidly connected to the outlet port, (2) filling the first reservoir with a fluid, wherein the fluid flows into the second reservoir from the first reservoir via the one-way valve; and (3) delivering the fluid through the outlet port.

In one aspect, the fluid comprises a drug (e.g., an olimus drug including at least one of sirolimus, everolimus, zotarolimus, tacrolimus, pimecrolimus, temsirolimus, ridaforolimus, biolimus or other sirolimus analogs).

In another aspect, the pressure in the first reservoir and the second reservoir is reduced after the fluid is delivered through the outlet port. In a further aspect, the volume of fluid in the first reservoir and the second reservoir is reduced after the fluid is delivered through the outlet port to, for example, a patient.

In one aspect, external pressure is applied to the first pressure receiver when the fluid volume moving from the second reservoir through the outlet port is below a volume flow threshold, causing fluid to flow into the second reservoir via the one-way valve.

In a further aspect, applying external pressure from a source external to the first reservoir to the first pressure receiver increases pressure in the first reservoir, thereby applying external pressure from the first reservoir to the second pressure receiver and increasing pressure in the second reservoir.

In this aspect, the second reservoir can remain pressurized after receiving fluid from the first reservoir. In another aspect, the second reservoir can deliver fluid through the outlet port when the pressure in the first reservoir decreases. In yet another aspect, the fluid volume in the first reservoir is refilled through the inlet port when the fluid volume in the first reservoir is below a second fluid volume threshold.

In one aspect, the rate of delivery of fluid through the outlet port is from about 0.01 μl/hour to about 100 μl/hour.

In a further aspect, external pressure is applied to the first pressure receiver of the first reservoir when the rate of fluid delivery through the outlet port is reduced by greater than about 10%.

In yet another aspect, the first pressure receiver is a first flexible membrane and the second pressure receiver is a second flexible membrane. The first flexible membrane can comprise a wall of the first reservoir and the second flexible membrane can comprise a wall of the second reservoir.

In a further aspect, manual compression force can optionally be applied to the first flexible elastic or inelastic collapsible pressure receiver, and compression force can be applied to the first or the second flexible pressure receiver by one or more forces derived from elastic membrane contraction, spring expansion, magnetic repulsion, compressed gas expansion, spring contraction, and magnetic attraction applied to a flexible membrane.

Aspects described herein provides a refillable, compression-activated pump for delivery of a fluid comprising a first reservoir, a second reservoir, a one-way valve, an inlet port, and an outlet port, wherein the first reservoir and the second reservoir are fluidly connected via the one-way valve, the first reservoir comprising the inlet port and a first pressure receiver, and the second reservoir comprising a second pressure receiver and the outlet port.

In some instances, the first pressure receiver comprises an elastic material or a non-elastic collapsible material. In further aspects, the second pressure receiver comprises an elastic material or a non-elastic collapsible material.

In some instances, the first reservoir can be filled with a fluid through the inlet port causing the fluid to move through the one-way valve into the second reservoir.

The fluid from the second reservoir can be delivered to a patient through the outlet port. In one aspect, a fluid volume in the first reservoir can be increased by supplying the fluid through the inlet port.

Increasing a pressure in the first reservoir by applying external pressure to the first pressure receiver can cause the fluid to move through the one-way valve into the second reservoir. Increasing the pressure in the first reservoir can apply the pressure to the second pressure receiver, increasing a pressure in the second reservoir. The pressure in the first reservoir can be increased when the pressure in the second reservoir is below a threshold sufficient to deliver the fluid from the second reservoir at a desired rate.

The first pressure receiver can be a first flexible membrane and the second pressure receiver can be a second flexible membrane. In one aspect, the first flexible membrane comprises a wall of the first reservoir. In another aspect, the second flexible membrane comprises a wall of the second reservoir.

The pump can include a fill stop (e.g., porous or non-porous) disposed above the second flexible membrane to limit the size of the second reservoir as the second reservoir expands. A first compression force can be applied to the first pressure receiver, and a second compression force can be applied to the second pressure receiver by one or more forces derived from elastic membrane contraction, spring expansion, magnetic repulsion, compressed gas expansion, spring contraction, and magnetic attraction applied to a flexible membrane. The first compression force can be a manual compression.

In another aspect, the pump further comprising a spring (e.g., helical spring, a torsional spring, and a lever spring) engaged with the second reservoir. Increasing a pressure in the first reservoir by applying external pressure to the first pressure receiver can lengthen the spring and increase a volume of the second reservoir. In this aspect, the fluid can move through the one-way valve into the second reservoir.

When force is applied to the second pressure receiver by a retraction of the spring, the fluid moves from the second reservoir into the outlet port. In some instances, an initial length of the spring is about equal to a diameter of the pump. The volume of the second reservoir can increased by about 10 to about 20% after applying external pressure to the first pressure receiver.

In some instances, a compression band (e.g., circular spiral spring, a compression spring, and a constant force spring) is disposed around the second reservoir. In this aspect, increasing a pressure in the first reservoir by applying external pressure to the first pressure receiver increases a circumference of the compression band and increases a volume of the second reservoir. The fluid can move through the one-way valve into the second reservoir.

When force is applied to the second pressure receiver by a retraction of the compression band, the fluid can be delivered through the outlet port from the second reservoir. In some instances, an initial circumference of the compression band is about equal to a diameter of the pump. In one aspect, the volume of the second reservoir is increased by about 10 to about 20% after applying external pressure to the first pressure receiver. In some instances, the refill port associated with the first reservoir for refilling the pump with the fluid.

The fluid can include a drug (e.g., olimus drug comprising at least one of sirolimus, everolimus, zotarolimus, tacrolimus, pimecrolimus, temsirolimus, ridaforolimus or biolimus).

Aspects described herein provide a kit comprising the pump aspects described herein, and a container comprising a drug (e.g., olimus drug comprising at least one of sirolimus, everolimus, zotarolimus, tacrolimus, pimecrolimus, temsirolimus, ridaforolimus or biolimus). The container can be selected from a group consisting of a vial, an ampule, a capsule, and a syringe.

Further aspects provide methods of controlled delivery of a fluid (e.g., to a subject or patient) by providing a pump having a first reservoir, a second reservoir, a one-way valve, an inlet port, and an outlet port. The first reservoir and the second reservoir can be fluidly connected via the one-way valve. The first reservoir can comprise the inlet port and a first pressure receiver. The second reservoir can comprise a second pressure receiver fluidly connected to the outlet port. The first reservoir can be filled with a fluid, wherein the fluid flows into the second reservoir from the first reservoir via the one-way valve, and the fluid is delivered through the outlet port.

The fluid can comprise a drug (e.g., olimus drug comprising at least one of sirolimus, everolimus, zotarolimus, tacrolimus, pimecrolimus, temsirolimus, ridaforolimus or biolimus). The fluid is delivered to a subject or patient. The pressure in the first reservoir and the second reservoir can be reduced after the fluid is delivered through the outlet port.

The volume of the fluid in the first reservoir and the second reservoir can be reduced after the fluid is delivered through the outlet port. The first reservoir can be refilled with at least a second volume of fluid after the fluid is delivered through the outlet port.

In some instances, external pressure is applied to the first pressure receiver when a fluid volume moving from the second reservoir through the outlet port is below a volume flow threshold, causing the fluid to flow into the second reservoir via the one-way valve.

In some instances, applying an external pressure from a source external to the first reservoir to the first pressure receiver increases the pressure in the first reservoir and applies an external pressure from the first reservoir to the second pressure receiver, wherein the pressure in the second reservoir is increased. The second reservoir can remain pressurized after receiving the fluid from the first reservoir. The second reservoir can deliver the fluid through the outlet port when the pressure in the first reservoir decreases.

In some instances, a fluid volume in the first reservoir is refilled through the inlet port when the fluid volume in the first reservoir is below a second fluid volume threshold. In some instances, a rate of delivery of the fluid through the outlet port is from about 0.01 μl/hour to about 100 μl/hour.

In a further aspect, external pressure is applied to the first pressure receiver of the first reservoir when a rate of fluid delivery through the outlet port is reduced by greater than about 10%. In this aspect, the first pressure receiver is a first flexible membrane and the second pressure receiver is a second flexible membrane.

The first flexible membrane can a wall of the first reservoir. The second flexible membrane can comprise a wall of the second reservoir.

In some instances, a porous (or non-porous) fill stop can be disposed above the second flexible membrane to limit an expansion of the second flexible membrane.

In some instances, a first compression force can be applied to the first pressure receiver, and a second compression force can be applied to the second pressure receiver by one or more forces derived from elastic membrane contraction, spring expansion, magnetic repulsion, compressed gas expansion, spring contraction, and magnetic attraction applied to a flexible membrane.

In some instances, the first compression force is a manual compression.

In further aspects, the pump further comprises a spring (e.g., helical spring, a torsional spring, and a lever spring) engaged with the second reservoir. In this aspect, a pressure in the first reservoir can be increased by applying external pressure to the first pressure receiver wherein the spring lengthens and a volume of the second reservoir is increased. The fluid can move through the one-way valve into the second reservoir.

When force is applied to the second pressure receiver by a retraction of the spring, fluid moves from the second reservoir into the outlet port. An initial length of the spring can be about equal to a diameter of the pump. The volume of the second reservoir can be increased by about 10 to about 20% after applying external pressure to the first pressure receiver.

In further aspects, the pump comprises a compression band (e.g., circular spiral spring, a compression spring, and a constant force spring) disposed around the second reservoir. In this aspect, when a pressure in the first reservoir is increased by applying external pressure to the first pressure receiver, the circumference of the compression band increases and a volume of the second reservoir increases. The fluid can then move through the one-way valve into the second reservoir.

When a force is applied to the second pressure receiver by a retraction of the compression band, the fluid moves into the outlet port from the second reservoir. An initial circumference of the compression band can be about equal to a diameter of the pump.

In this aspect, the volume of the second reservoir can be increased by about 10 to about 20% after applying external pressure to the first pressure receiver.

The pump can further comprise a refill port associated with the first reservoir for refilling the pump with the fluid. The fluid can comprise a drug (e.g., olimus drug comprising at least one of sirolimus, everolimus, zotarolimus, tacrolimus, pimecrolimus, temsirolimus, ridaforolimus or biolimus).

Any suitable drug or combination of drugs can be used to fill the compression-activated pumps described herein, including an olimus drug, (e.g., at least one of sirolimus, everolimus, zotarolimus, tacrolimus, pimecrolimus, temsirolimus, ridaforolimus or biolimus, or other sirolimus analogs).

Additional drugs that can be used alone or in combination with other drugs include at least one of the following drugs: actimmune, paclitaxel, brentuximab, vedotin, pemetrexed, bevacizumab, pegylated liposomal, doxorubicin, carboplatin, cisplatin, oxaliplatin, cetuximab, gemcitabine, eribulin, mesylate, trastuzumab, cabazitaxel, emtansine, pembrolizumab, carfilzomib, nivolumab, pertuzumab, rituximab, paclitaxel, docetaxel, temsirolimus, bendamustine, panitumumab, bortezomib, venofer, zoledronic acid, thiazolidinediones, glipizide, glimepiride, metformin, victoza, or jardiance; at least one chemotherapy drug; at least one pain reliever; at least one nutrient; or at least one agent to treat at least one of diabetes, arthritis, cancer, dehydration, or a migraine.

Additional classes of drugs that can used in aspects described herein include, but are not limited to antiplatelets, antithrombins, anticoagulants, cytostatic agents, cytotoxic agents, antiproliferative agents, vasodilators, alkylating agents, antimicrobials, antibiotics, antimitotics, anti-infective agents, antisecretory agents, anti-inflammatory agents, immunosuppressive agents, antimetabolite agents, growth factor antagonists, free radical scavengers, antioxidants, radiotherapy agents, anesthetics, radiopaque agents, radiolabeled agents, nucleotides, cells, proteins, glycoproteins, hormones, anti-stenosis agents, anti-fibrotic agents, isolates, enzymes, monoclonal antibodies, ribonucleases and any combinations thereof.

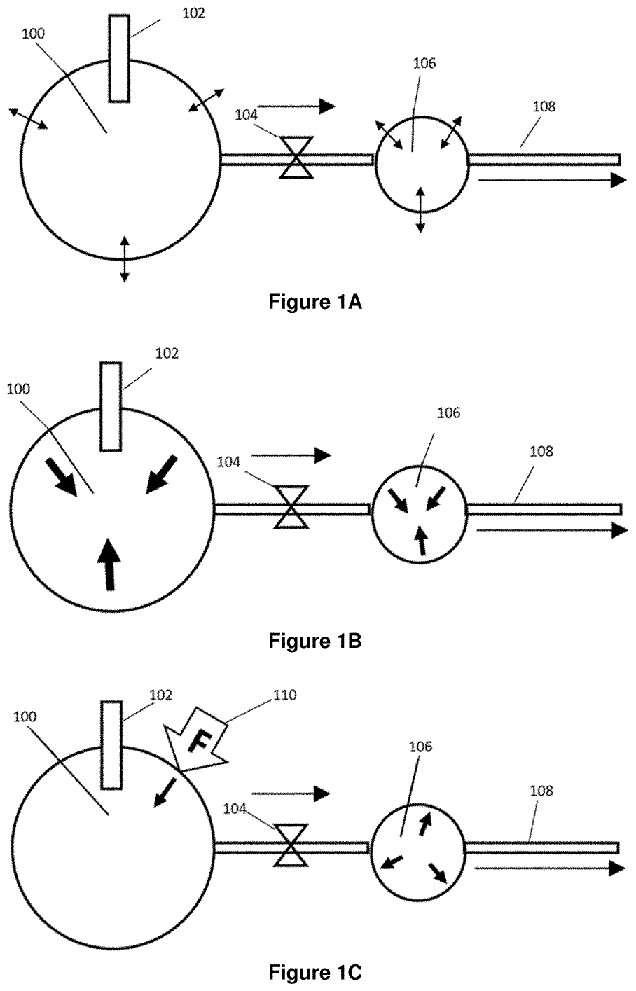

With reference to FIG. 1A, an exemplary compression-activated, elastic refillable pump is shown in cross-section with first reservoir 100, fill/refill port 102, one-way valve 104, second reservoir 106, and outlet port 108. In this aspect, drug can be delivered to first reservoir 100 by any suitable means (e.g., syringe and needle, catheter or other tubing). When first reservoir 100 is depleted of drug fluid, it can be refilled through fill/refill port 102. Arrows show that both reservoirs can expand or shrink.

It is understood that first reservoir 100 can be made of any suitable elastic material (e.g., silicone rubber, polyurethane) that can be fluidly connected to fill/refill port 102 and one-way valve 104 without, for example, significant leaking of drug fluid. Fill/refill port 102 can be made of, for example, any suitable puncturable diaphragm material. It is also understood that the exemplary compression-activated, elastic refillable drug delivery device can be small enough to be implanted into a patient, worn on the patient's body, or carried in clothing or another accessory.

FIG. 1B shows the exemplary compression-activated, elastic refillable drug pump of FIG. 1A after drug has moved from first reservoir 100 to second reservoir 106 through one-way valve 104 and through outlet port 108 to a patient (not shown). The arrows depict the shrinking of first reservoir 100 and second reservoir 106 as the drug fluid is depleted.

FIG. 1C shows compression force (F) being applied to first reservoir 100 to restore pressure to first reservoir 100 and deliver drug through one-way valve 104 to second reservoir 106. Compression force (F) can be applied by pressing first reservoir 100 with a finger or any other suitable device. Compression force (F) can be directly applied to first reservoir 100 or indirectly applied through, for example, skin or clothing. Compression force (F) can be applied periodically (e.g., every 1, 2, 4, 6, 12, 24, 48, 72, or 96 hours, weekly, monthly) depending on the desired rate and volume of drug delivery. It should be understood that compression force (F) can be applied from any suitable direction as long as first reservoir 100 is compressed in a manner that can restore the desired pressure to second reservoir 106.

First reservoir 100 can optionally have a designated area for optimal application of compression force (F). The designated area can be adapted to physically accommodate, for example, a finger or other device. In this aspect, a patient or caregiver can readily find the optimal area to apply force using the sense of touch without the need for visual confirmation.

First reservoir 100 and second reservoir 106 can optionally each include one or more sensors to detect the pressure and fluid volume in the reservoir. These sensors can provide audible or visual feedback to a user or caregiver and can optionally be configured with a wireless device or connection to a device (e.g., computer, phone, tablet, etc.) and alert the user or caregiver if the pressure or volume in the reservoir is above or below a desired level. Further sensors can be provided to measure the amount of drug delivered to the patient over a desired period of time and alert the patent or caregiver when the first reservoir needs to be refilled.

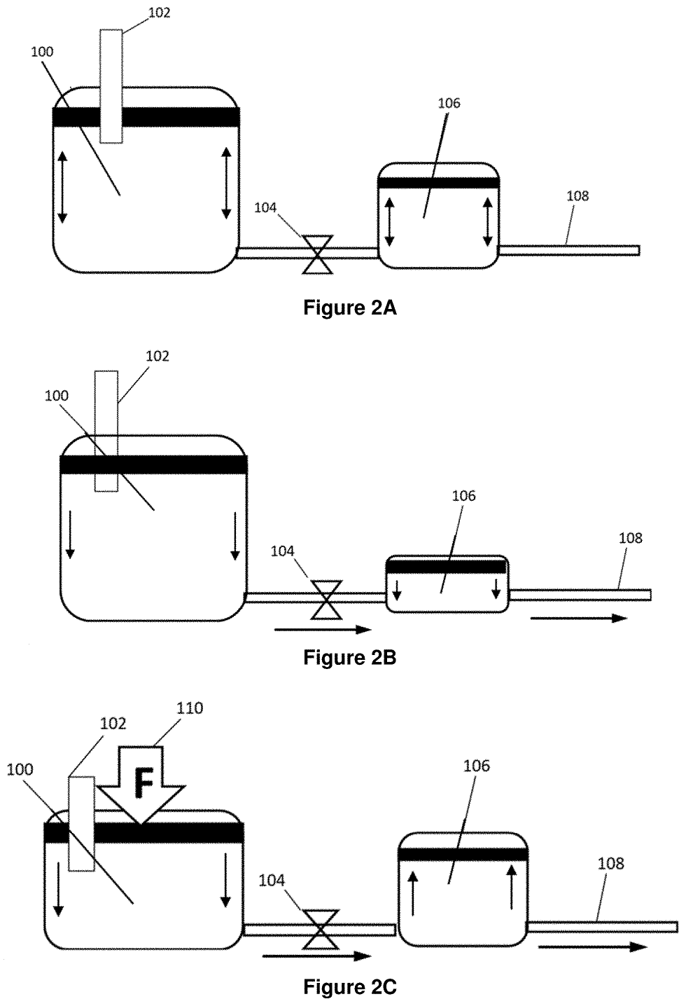

FIG. 2A shows an exemplary compression-activated, non-elastic, collapsible, refillable drug pump in cross-section with first reservoir 100, fill/refill port 102, one-way valve 104, second reservoir 106, and outlet port 108. In this aspect, drug can be delivered to first reservoir 100 by any suitable means (e.g., syringe and needle, catheter or other tubing). When first reservoir 100 is depleted of drug fluid, it can be refilled through fill/refill port 102.

It is understood that first reservoir 100 can be made of any suitable non-elastic collapsible, material (e.g., polyethylene, polytetrafluoroethylene) that can be fluidly connected to fill/refill port 102 and one-way valve 104 without, for example, significant leaking of drug fluid. Fill/refill port 102 can be made of, for example, diaphragm materials, as noted above. It is also understood that the exemplary compression-activated, non-elastic refillable drug delivery device can be small enough to be implanted into a patient, worn on the patient's body, or carried in clothing or another accessory.

FIG. 2B shows the exemplary pump of FIG. 2A after drug has moved from first reservoir 100 to second reservoir 106 through one-way valve 104 and through outlet port 108 to a patient (not shown). Second reservoir 106 is shown as collapsing as the drug fluid is depleted following delivery of drug to the patient (arrows).

In FIG. 2C, compression force (F) is applied to first reservoir 100 of the exemplary compression-activated, non-elastic, refillable drug delivery device of FIG. 2B and restores drug to second reservoir 106. Arrows show the volume of the first reservoir shrinking, and the volume of the second reservoir expanding while the force is applied.

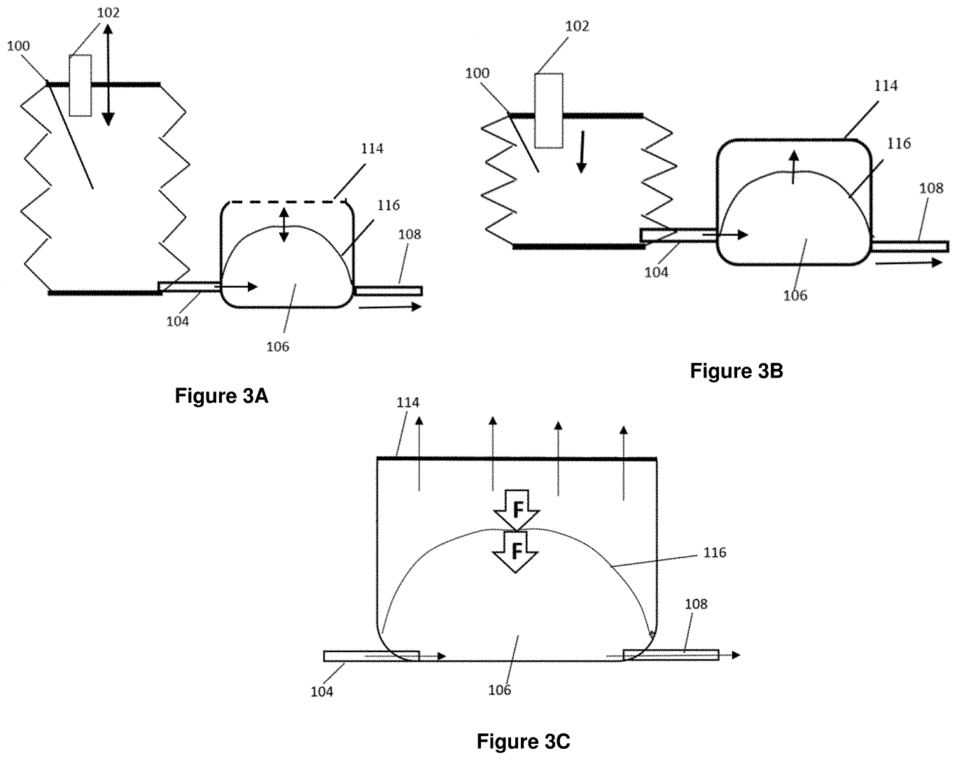

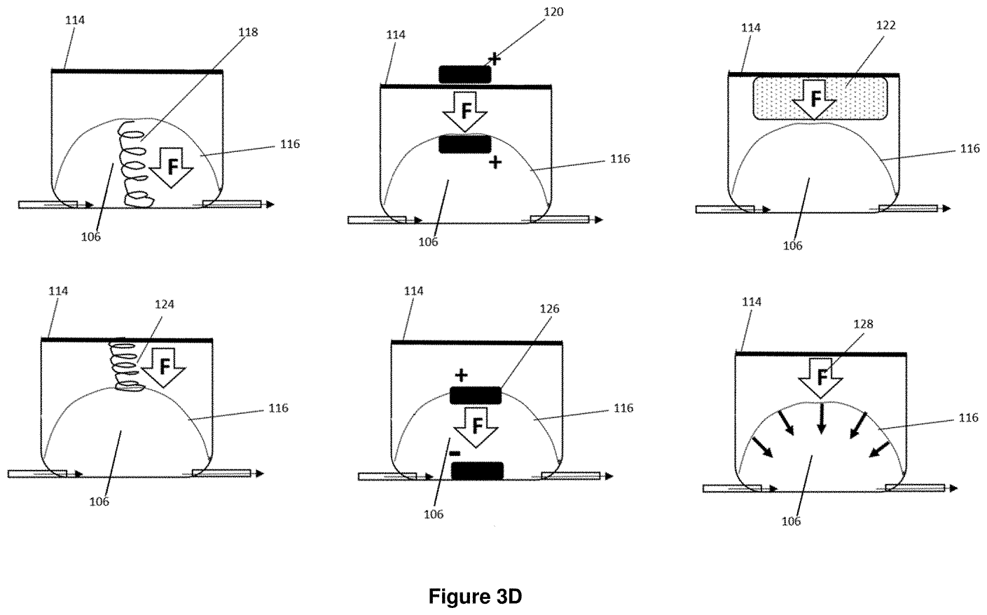

FIG. 3A shows an aspect of a compression-activated, refillable drug pump where first reservoir 100 is non-elastic and collapsible (made of a flexible sack, bellows etc.) and the second reservoir contains an elastic membrane. Second reservoir 106 features porous fill stop 114 and membrane 116. As shown in FIG. 3B, the second reservoir 106 expands when compression force is applied to the first reservoir 100. In this aspect, porous fill stop 114 is disposed above membrane 116 to limit the maximum size of second reservoir 106 as second reservoir 106 expands.

As shown in FIG. 3C, the compression force (F) applied to membrane 116 drives drug delivery through outlet 108. Fill stop 114 confines the volume of second reservoir 106. Membrane 116 can be, for example, an elastic membrane or other collapsible element. In this aspect, the rate of drug delivery is controlled by the pressure within second reservoir 106 and the resistance in the tubing of outlet port 108.

FIG. 3D shows exemplary aspects of mechanisms to apply force to membrane 116, which may be an elastic or inelastic collapsible membrane, including (1) spring expansion, (2) magnetic repulsion, (3) compressed gas expansion, (4) spring contraction, and (5) magnetic attraction force. FIG. 3D also shows an elastic membrane exerting (6) compression force on reservoir 106.

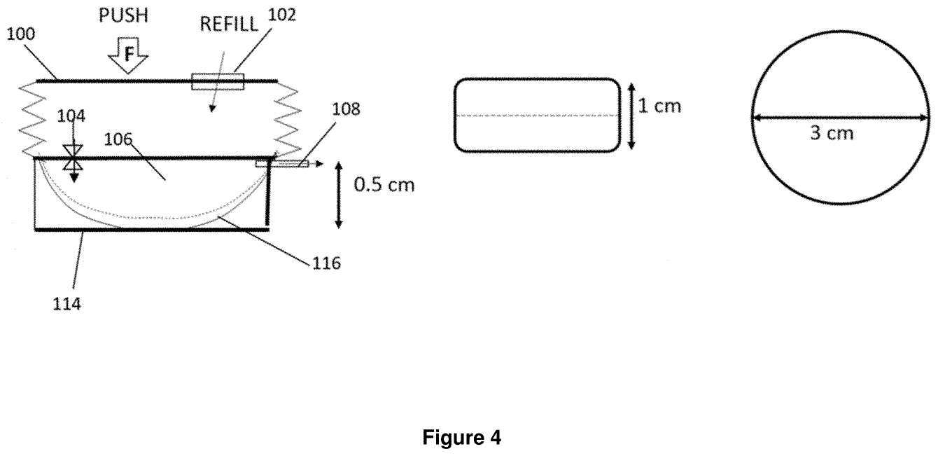

FIG. 4 shows an exemplary aspect where the volume of the second reservoir 106 shrinks as the height of the spherical cap membrane 116 shrinks by 10% (from a total height of 5 mm to 4.5 mm, to the dotted line in FIG. 4). In this aspect, the pressure in second reservoir 106 drops by about 20%, and the drug delivery rate drops by about 20%. The total volume delivered in this aspect is 195 μl. If the desired delivery rate is 1000 ul/month (36 μl/day×28 days), compression force can be applied every 5.4 days to maintain a delivery rate of 36 μl per day (195 μl every 5.4 days). Afterwards, reservoir 100 can be reloaded, for example, approximately every month. Compression force applied to first reservoir 100 will then re-pressurize second reservoir 106.

In the aspect of FIG. 4, drug fluid can be provided to first reservoir 100 through inlet port 102. Inlet port 102 can also be used to refill first reservoir 100 when drug fluid is depleted. Drug fluid can flow through one-way valve 104 into second reservoir 106. In this aspect, the side walls of first reservoir 100 are collapsible which provides flexibility that facilitates applying a compression force (F) to re-establish a desired pressure in first reservoir 100. Drug fluid in second reservoir 106 can be delivered through outlet port 108 due to pressure and volume in second reservoir 106. Exemplary dimensions for first reservoir 100 and second reservoir 106 (e.g., total combined height of 1 cm) are shown as is an exemplary diameter of 3 cm for the two reservoir pump device.

In this aspect, (i.e., FIG. 4) the drug flow rate would vary between about 30-40 ul per day (about 1 ml/month). Since the volume of reservoir 100 is about 3.5 ml in this aspect, the pump would need to be refilled approximately every 3 months. It is understood that in this aspect, the overall pump size and operational parameters can be configured and optimized for a desired application and drug delivery rate by varying the size and volume of reservoirs 100 and 106, and by a choice of mechanisms used to apply compression force (e.g., FIG. 3D).

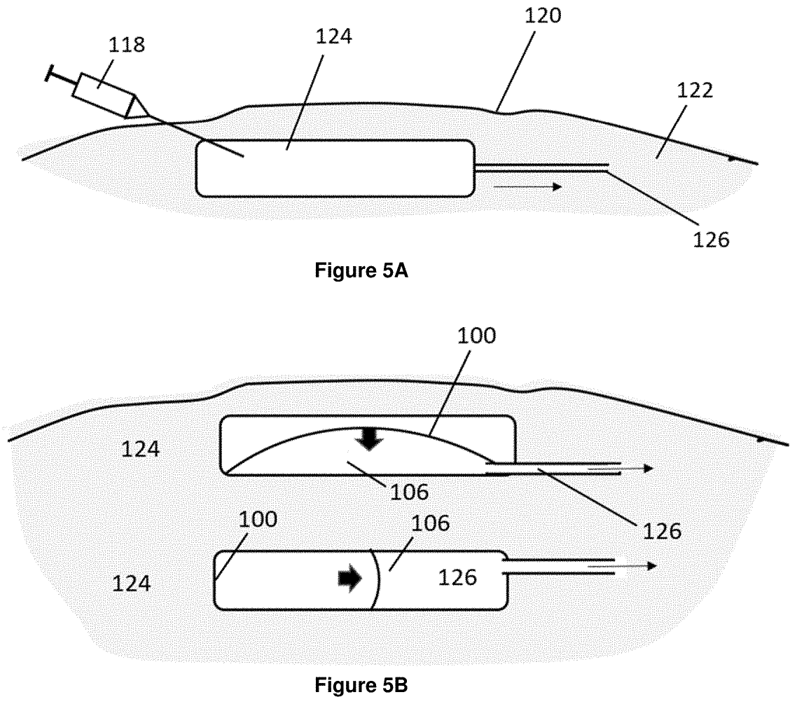

FIG. 5A shows an exemplary implanted horizontal subcutaneous compression pump 124 being filled using syringe 118 filled with a drug (not shown) piercing skin 120 and tissue 122. As described herein, pump 124 can be configured, for example, to deliver drug through outlet port 126 to a drug-eluting cuff, graft or other target or device.

FIG. 5B shows two exemplary configurations of a drug pump in accordance with aspects described herein. The top panel shows the exemplary configuration depicted in FIGS. 2-4 where an axial force (arrow) is applied to drug pump 124 through first reservoir 100 and second reservoir 106 compressing the membrane of second reservoir 106 and pushing drug out of outlet port 126. The bottom panel illustrates an exemplary configuration where a longitudinal force (arrow) is applied to second reservoir 106 (e.g., by applying an axial force to first reservoir 100) which expands the membrane of first reservoir 100 and pushes drug out of outlet port 126.

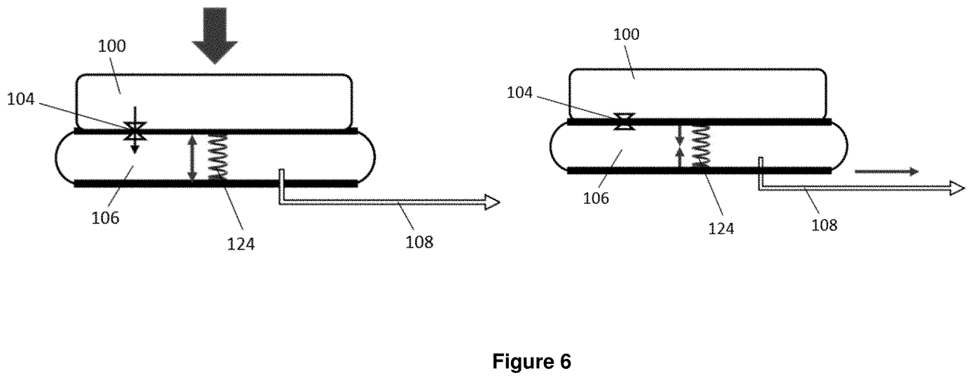

FIG. 6 shows an exemplary axial pump configuration having first reservoir 100 and second reservoir 106 connected by one-way valve 104 as previously described. Spring 124 is configured to be disposed in second reservoir 106 to provide reciprocating resistance force when a compression force (arrow) is applied to first reservoir 100. In this example, the diameter of second reservoir 106 is greater than first reservoir 100. However, it will be appreciated that the relative diameters of first reservoir 100 and second reservoir 106 can be adjusted depending on the desired application.

In operation, as shown in the left panel of FIG. 6, applying a force to first reservoir 100 causes a fluid (e.g., containing a drug) in first reservoir 100 to move through one-way valve 104 into second reservoir 106, causing spring 124 to lengthen, and expanding the volume of second reservoir 106. In one example, first reservoir 100 can be refilled through a refill port (not shown) when spring 124 has compressed about 10% of the thickness in second reservoir 106 and depleted about 10% of the fluid in second reservoir 106 (FIG. 6, right panel).

It will be appreciated that first reservoir 100 can be refilled when spring 124 has compressed about 10, 20, 30, 40, 50, 60, 70, 80, 90% etc. of the thickness in second reservoir 106, and about, for example, 10% of the fluid in second reservoir 106 has been depleted. In this example of a “vertical compression pump,” pushing down on the top results in equal force pushing up on the bottom of the pump. In one example, spring 124 can operate in the range of about 27-30 mm and can expand-contract in the range of about 4.5-5.0 mm.

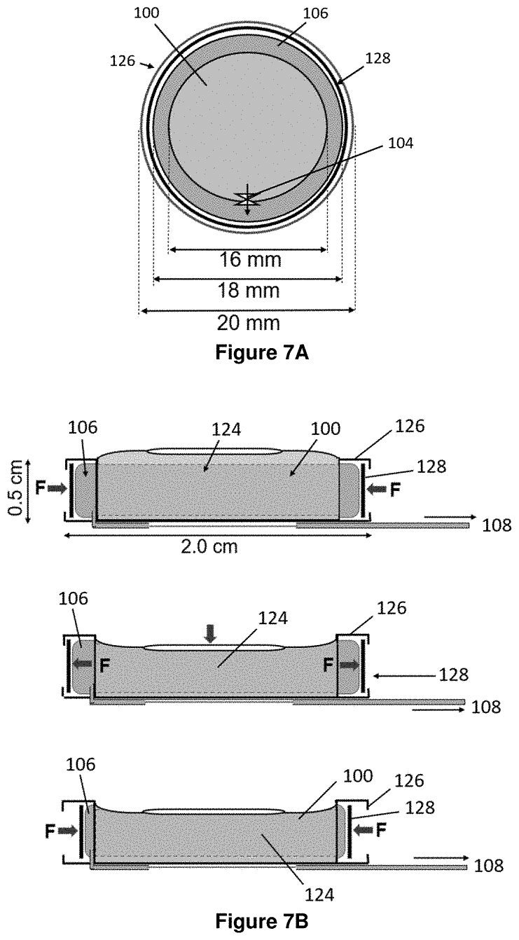

FIG. 7A illustrates a top view of an exemplary longitudinal force pump configuration having optional casing 126 encompassing compression band 128, second reservoir 106, and first reservoir 100. First reservoir 100 and second reservoir 106 are connected by one-way valve 104.

FIG. 7B shows a side view of the exemplary longitudinal force pump configuration of FIG. 7A in three states—prior to applying longitudinal force (arrow) to pump 124 (top panel), during the application of longitudinal force (middle panel), and after applying longitudinal force (bottom panel). In this aspect, when axial force is applied to first reservoir 100, first reservoir 100 expands and applies a longitudinal force to second reservoir 106, compressing second reservoir 106 against compression band 128, and pushing drug from second reservoir 106 through outlet port 108.

The refillable, pressure activated pump as described herein can be configured to be implanted in a patient to deliver a desired drug in the drug fluid patient over a time period ranging from minutes to hours to days to months. In each case, the size of the pump can be configured to contain the amount of drug desired to be delivered over an initial period of time. The implanted pump can remain in a patient and refilled as described herein for repeated delivery of a drug at a desired delivery rate. The pump can be configured with sensors to detect the volume and pressure in the reservoirs of the pump and alert the patient or health care provider that the volume or pressure in the pump needs to be adjusted.

### REFERENCES

(1) U.S. Pat. No. 5,399,352

(2) U.S. Pat. No. 8,808,255

(3) U.S. Pat. No. 8,721,711

What is claimed is:

1. A refillable, compression-activated pump for delivery of a fluid comprising:
a first reservoir, a second reservoir, a one-way valve, an inlet port, and an outlet port, wherein the first reservoir and the second reservoir are fluidly connected via the one-way valve, the first reservoir comprising the inlet port and a first pressure receiver, and the second reservoir comprising a second pressure receiver and the outlet port.

2. The pump of claim 1, wherein the first pressure receiver is a first flexible membrane and the second pressure receiver is a second flexible membrane.

3. The pump of claim 2, wherein the first flexible membrane comprises a wall of the first reservoir.

4. The pump of claim 3, wherein the second flexible membrane comprises a wall of the second reservoir.

5. The pump of claim 4, further comprising a porous fill stop disposed above the second flexible membrane to limit the size of the second reservoir as the second reservoir expands.

6. The pump of claim 2, further comprising a spring engaged with the second reservoir.

7. The pump of claim 2, further comprising a compression band disposed around the second reservoir.

8. The pump of claim 1, further comprising a refill port associated with the first reservoir for refilling the pump with the fluid.

9. The pump of claim 8, wherein the fluid comprises a drug.

10. The pump of claim 9, wherein the drug is an olimus drug comprising at least one of sirolimus, everolimus, zotarolimus, tacrolimus, pimecrolimus, temsirolimus, ridaforolimus or biolimus.

11. A method of controlled delivery of a fluid comprising:
providing a pump having a first reservoir, a second reservoir, a one-way valve, an inlet port, and an outlet port, wherein the first reservoir and the second reservoir are fluidly connected via the one-way valve, the first reservoir comprises the inlet port and a first pressure receiver, and the second reservoir comprises a second pressure receiver is fluidly connected to the outlet port;
filling the first reservoir with a fluid, wherein the fluid flows into the second reservoir from the first reservoir via the one-way valve; and
delivering the fluid through the outlet port.

12. The method of claim 11, wherein the fluid comprises a drug.

13. The method of claim 12, wherein the drug is an olimus drug comprising at least one of sirolimus, everolimus, zotarolimus, tacrolimus, pimecrolimus, temsirolimus, ridaforolimus or biolimus.

14. The method of claim 11, wherein the pump further comprises a spring engaged with the second reservoir.

15. The method of claim 14, further comprising increasing a pressure in the first reservoir by applying external pressure to the first pressure receiver wherein the spring lengthens and a volume of the second reservoir is increased, wherein the fluid moves through the one-way valve into the second reservoir, and wherein when force is applied to the second pressure receiver by a retraction of the spring, fluid moves from the second reservoir into the outlet port.

16. The method of claim 15, wherein a volume of the second reservoir is increased by about 10 to about 20% after applying external pressure to the first pressure receiver.

17. The method of claim 11, wherein the pump further comprises a compression band disposed around the second reservoir.

18. The method of claim 17, further comprising increasing a pressure in the first reservoir by applying external pressure to the first pressure receiver, wherein a circumference of the compression band increases and a volume of the second reservoir increases, wherein the fluid moves through the one-way valve into the second reservoir, and wherein when a force is applied to the second pressure receiver by a retraction of the compression band, the fluid moves into the outlet port from the second reservoir.

19. The method of claim 18, wherein a volume of the second reservoir is increased by about 10 to about 20% after applying external pressure to the first pressure receiver.

20. A kit comprising the pump of claim 1, and a container comprising a drug.
